(19) United States
(12) Patent Application Publication
(10) Pub. No.: US 2017/0072024 A1
(43) Pub. Date: Mar. 16, 2017

(54) ANTIMICROBIAL COMPOSITION COMPRISING A CARBOHYDRATE, GLUCOSE OXIDASE AND ZINC OXIDE

(71) Applicant: MELIPHARM, Limoges (FR)

(72) Inventors: Carmen MALEPEYRE, VERNEUIL SUR VIENNE (FR); Anais RAYNAUD, LES PALAIS SUR VIENNE (FR); Fabien QUERO, LE BOUSCAT (FR)

(21) Appl. No.: 15/310,974

(22) PCT Filed: May 13, 2015
(86) PCT No.: PCT/FR2015/051270

§ 371 Date: Nov. 14, 2016

(30) Foreign Application Priority Data
May 14, 2014 (FR) 1454293

(51) Int. Cl. A61K 38/44; A61K 33/30; A61K 36/42; A61K 9/00; A61K 36/70; A61K 31/7004; A61K 35/644

(52) U.S. Cl. CPC A61K 38/443; A61K 31/7004; A61K 33/30; A61K 35/644; A61K 9/0014; C12Y 101/03004; A61K 36/70; A61K 36/42

(57) ABSTRACT

A composition that includes a mixture made up of at least one carbohydrate, glucose oxidase and zinc oxide. This topical antimicrobial composition is particularly useful to care for and treat affections of the skin and mucous membranes.

### FIELD OF THE INVENTION

The present invention relates to a composition comprising a mixture of at least one carbohydrate, glucose oxidase and zinc oxide as an antimicrobial composition, and its use on the skin or mucus membranes.

### BACKGROUND OF THE INVENTION

It is quite often necessary to use topical antimicrobial compositions, in particular to treat wounds, or to treat cutaneous microbial infections or microbial pathologies of the skin. Known products used to treat wounds in particular include creams, silver or iodine pomades or dressings, or honey-based dressings.

Silver dressings are often quite expensive and generally are cytotoxic for cells in the dermis and epidermis (Landsdown ABG.A pharmacological and toxicological profile of silver as an antimicrobial agent in medical devices. Adv Pharm Sci 2010; 2010:910686.Epub 2010 Aug. 24, Burd A, Kwok C H, Hung S C, et al. A comparative study of the cytotoxicity of silver-based dressings in monolayer cell, tissue explant, and animal models. Wound Repair Regen 2007; 15(1):94-104) and when used in pediatrics, they may cause elevated silver levels in the blood.

Iodine dressings are also cytotoxic, and their antimicrobial activity decreases considerably in the presence of organic materials (pus, fibrin or necrosis). Furthermore, there is a risk of sensitivity in contact with eczema and allergy to these iodine-based products.

In recent years, honey has become a product of interest in the medical field to treat certain infections in wounds; however, the antimicrobial properties of different honeys vary considerably depending on their origins. Yet honeys nevertheless have a significant antimicrobial activity that is often cytotoxic to skin cells. It is therefore very difficult to find a honey that is both highly antimicrobial and non-cytotoxic.

Applications WO-2005/112852, WO-2007/045931, US2004/121020 and WO-2004/000339 for example describe honey-based compositions, dressings or dressing derivatives with a base of manuka honey associated with texturing agents or other active ingredients. The described compositions make it possible to decontaminate wounds and thus favor their healing.

However, these compositions are not necessarily satisfactory in terms of efficacy, in particular on cell migration and proliferation, which is necessary for healing.

Furthermore, the manufacture of these compositions is often complex and expensive, in particular due to the use of complex manufacturing methods (association with interfaces) and/or the addition of active ingredients to specific and expensive honeys (for example, manuka honey).

In application FR 2,995,532, honey-based compositions are described that offset these drawbacks and which, aside from their antibacterial efficacy, are capable of inhibiting biofilm formation, having a bactericidal effect with respect to biofilms that have already formed, and preventing adhesion of the bacteria.

### BRIEF SUMMARY OF THE INVENTION

The aim of the present invention is to propose a different antimicrobial composition that is at least as efficacious as those described in application FR 29,995,532.

To that end, the invention relates to a composition comprising a mixture made up of at least one carbohydrate, glucose oxidase and zinc oxide, in particular for use as a topical antimicrobial product.

The mixture according to the invention has a significant and surprising synergistic effect with respect to the known compositions, which makes it possible to propose a composition having an antimicrobial activity exceeding that of the current honey-based products, with no variation in cytotoxicity.

Other features and advantages will emerge from the following detailed description of the invention.

### DETAILED DESCRIPTION OF THE INVENTION

The invention therefore targets a composition comprising a mixture made up of at least one carbohydrate, glucose oxidase and zinc oxide.

The carbohydrate is very preferably at least glucose. Preferably, it is glucose alone or a mixture of carbohydrates in particular containing glucose.

The carbohydrate source may in particular be honey. The honey may be natural or artificial honey or a mixture of several natural and/or artificial honeys.

“Artificial honey” refers to at least one sugar. It may be a combination of at least two sugars.

In the event the carbohydrate source is natural honey, it may preferably be thyme honey and/or honeydew honey and/or buckwheat honey and/or manuka honey and/or mixtures thereof. It may for example be a honey as described in application FR 1,258,722.

In the event the carbohydrate source is artificial honey, it is preferably primarily made up of glucose and/or fructose and/or sucrose.

According to one particularly suitable embodiment, the carbohydrate is glucose.

The carbohydrate(s) preferably make up 1 wt % to 99 wt % of the total weight of the composition, still more preferably between 1 and 60 wt %.

In addition to the carbohydrate(s), the composition according to the invention may comprise glucose oxidase and zinc oxide.

Glucose oxidase is an enzyme that acts as a catalyst for the oxidation reaction of the glucose in hydrogen peroxide in the presence of water. This enzyme may be of natural or biotechnological origin. It is primarily present in microbial sources and obtained by fermentation of mushrooms, such as Aspergillus niger.

Glucose oxidase is present at a dose of at least 0.025 mU (milli-units) per gram of composition according to the invention, preferably between 0.5 mU and 50 U per gram of composition.

If the carbohydrate is honey, which can produce hydrogen peroxide because of the glucose oxidase that it naturally contains, the amount of glucose oxidase that it contains will not be sufficient to obtain the activity and the efficacy connected with the presence of glucose oxidase as defined in the invention. The addition of honey and other components with glucose oxidase at a dose of at least 0.025 mU is necessary to obtain the results described in the invention. In the case where the composition contains honey, the composition is supplemented with glucose oxidase, in addition to the composition that optionally can be contained in the honey and this additional dose is preferably at least 0.25 mU.

Zinc oxide is a mineral compound with formula ZnO used in many applications, in particular in topical preparations. For the present invention, the size of the zinc oxide particles used is very preferably smaller than 50 μm.

The zinc oxide preferably makes up between 0.1 wt % and 40 wt % of the total weight of the composition, still more preferably between 0.1 wt % and 10 wt %.

The composition according to the invention may be made up exclusively of carbohydrate(s), glucose oxidase and zinc oxide, or may contain other components.

In addition to the mixture, it may in particular also comprise one or more components chosen from among vanillin, caprylylglycol, pentylene glycol, 1,2-hexanediol, a propolis extract, methylglyoxal, glycerol, propylene glycol, polyethylene glycol, pectin, gums, carrageenan, xanthan, alginate salts, cellulose and its derivatives, starch and its derivatives, chitosan, triglycerides, vitamins, butters, waxes, glyceryl stearate, Vaseline, paraffins, kaolin, bentonite, lanolin.

It may also contain excipients chosen from among excipients that are dermatologically compatible and/or applicable on the skin and mucous membranes in order to obtain a composition in powder form, or in liquid form such as a lotion, or semi-solid such as a powder for cutaneous use, a spray, cream, pomade, gel, paste, emulsion, suppository or vaginal suppository. These may for example be excipients such as sugar and sugar derivatives, polysaccharides (pectin, starch and derivatives, alginate and derivatives, chitosan and derivatives, cellulose derivatives, gums, beta-glucans), synthesis polymers, waxes, natural or artificial oils, butters, mineral products (silica, talc, clays, titanium oxide), glycerides and other fatty esters, surface active agents, water, ethanol, propylene glycol, butylene glycol, glycerol, sorbitol, extracellular matrix compounds such as hyaluronic acid or collagen, hydrocarbons and silicones, proteins and peptides, and other excipients known by those skilled in the art.

The composition may also comprise auxiliary formulation additives such as surface active agents, gelling agents, absorbent agents, humectants, solvents, spreading agents, stabilizers, sequestering agents, rheological modifiers, preservatives, antioxidants and antimicrobials, dyes and scents.

The composition according to the invention may be sterile, i.e., it has undergone a sterilization process. The sterilization is preferably done by gamma radiation.

The composition according to the invention may be obtained by simple mixing of the components. Preferably, it is obtained by a homogenous powder mixture or by dispersion of powders in a honey composition.

The composition according to the invention may assume various powder or liquid or semi-liquid forms, in particular cutaneous powder, spray, lotion, cream, emulsion, gel, pomade, vaginal suppository, suppository.

The composition is used as a topical antimicrobial product, in particular a healthcare product or cosmetic composition. In fact, the mixture according to the invention acts synergistically to simultaneously:
limit microbial proliferation: the composition is capable of destroying germs on the skin, in particular in wounds,
inhibit biofilm formation on the skin and/or destroy biofilms that have already formed on the skin, in particular in wounds,
prevent or combat the adhesion of micro-organisms on the skin, in particular in wounds.

The decreased microbial load makes it possible to promote healing indirectly. Furthermore, the composition according to the invention may also make it possible to favor the proliferation of fibroblasts for a healing effect.

The effects obtained with the mixture of the three components of the composition according to the invention are much better than those obtained with each of the components alone or mixtures of each of the components in pairs. There is a surprising synergistic effect.

At the microbial level, each of the three components of the composition according to the invention may make it possible to limit microbial proliferation very slightly, but when they are used together, the decrease in microbial proliferation is greater than the sum of the limitations induced by the components alone, and there is a surprising synergistic effect.

According to the invention, the composition may therefore advantageously be used as a healthcare product for topical application on the skin or mucous membranes in humans or animals, very preferably as:
health product, in particular a medical device for therapeutic purposes designed to be applied on irritations or skin lesions, in particular to:
   treat first and second degree burns,
   treat and/or prevent postoperative healing disunions,
   treat and/or prevent postoperative residual cavities of the pilonidal sinuses,
   treat surgical scars infected after flattening,
   treat ulcers and eschars in the budding phase,
   treat dermabrasions, traumatic and/or surgical wounds,
   treat acute and chronic wounds,
   treat cancerous wounds,
   treat ostomy locations,
   treat superficial wounds,
composition intended to treat microbial irritations and/or infections, in particular scratch dermatitis, diaper rash, acne, dermatitis, impetigo, boils, folliculitis, whitlow and mycosis, in particular as a cosmetic composition,
oral gel, in particular to treat canker sores and mucositis,
lip stick or balm designed to treat cracking of the skin,
cream for the hands and feet in particular designed to treat cracks and chilblains,
balm intended for the prevention, care and/or treatment of cracked nipples,
vaginal gel to treat skin lesions and microbial infections,
suppository or rectal cream to treat rectal lesions and irritations.

A medical device for therapeutic purposes refers to a health product intended to be used in humans or animals in particular for the prevention, control, treatment and/or attenuation of a disease, injury, affection or cutaneous irritation.

If it is a health product for healing skin lesions in the form of a gel, cream or pomade, the composition according to the invention may in particular apply according to the following usage protocol:
rinse the wound and dry gently,
cover the entire wound or the secondary dressing with a film of the composition according to the invention,
cover with compresses and occlusive dressing during first applications for very wet wounds.

During the cleaning phase, and depending on the condition of the wound, it is advised to apply a new dressing one to two times every 24 hours.

During the budding phase, it is advised to apply a new wrapping every 48 to 72 hours.

During the epithelialization phase, it is recommended to apply a new dressing every 3 or 4 days.

The invention will now be illustrated through example compositions and results from trials demonstrating its efficacy.

### EXAMPLES OF COMPOSITION ACCORDING TO THE INVENTION

### Example 1

The composition of example 1 is a powder made up of:
98.9% Glucose
1% ZnO
0.1% Glucose oxidase

The composition is obtained by mixing the compounds.

### Example 2

The composition of Example 2 is a pomade made up of:
quantity sufficient for 100% mixture of natural honeys
25% triglycerides
11% white beeswax
7.5% shea butter
1.83% tocopheryl acetate
5% glyceryl monostearate
5% ZnO
0.01% Glucose oxidase
   the percentages being weight percentages relative to the total weight of the dry mass of the composition.

The composition is obtained by carrying out the following steps:
finely dispersing the powders in the honey,
heating the mixture to less than 40° C.,
heating the lipophilic phase (wax, butter and glycerides) to approximately 70° C. to melt the solids,
when the lipophilic phase has reached the temperature of 40° C., adding the tocopheryl acetate and the mixture of honeys and powders,
mixing the pomade until it cools.

### Example 3

The composition of example 3 is a paste made up of:
quantity sufficient for 100% artificial honey
2% ZnO
0.1% Glucose oxidase

The composition is obtained by mixing the compounds.

### Example 4

The composition of example 4 is a paste made up of:
quantity sufficient for 100% buckwheat honey
1% ZnO
0.1% Glucose oxidase

The composition is obtained by mixing the compounds at ambient temperature.

### Example 5

The composition of example 5 is a gel made up of:
quantity sufficient for 100% honeydew honey
15% Glycerol
17.5% PEG 1500
4% Pectin
10% ZnO
1.5% Glucose oxidase

The composition is obtained by:
mixing the powders, then at 40° C., adding the Glycerol and honey,
dissolving the PEG at 70° C.,
cooling to 40° C.,
adding the honey base into the PEG at 40° C., mixing until it cools. Evaluation of the efficacy of the composition according to the invention

Trials have been conducted to demonstrate the antimicrobial efficacy of several compositions according to the invention and to compare that efficacy to that of the components alone.

1. In Vitro Antibacterial and Anti-Biofilm Efficacy

To conduct the efficacy tests, several formulas were tested:
formula 1: Honey alone
formula 2: Honey+0.1% glucose oxidase
formula 3: ZnO alone 1%
formula 4: Honey+ZnO 1%
formula 5: Honey+0.1% glucose oxidase+1% ZnO (non-irradiated)
formula 6: Honey+0.1% glucose oxidase+1% ZnO+excipients (formula with normal aging=4 months; irradiated)
formula 7: Honey+0.1% glucose oxidase+1% ZnO+excipients (formula with accelerated aging 92 days=1 year; irradiated)
formula 8: glucose oxidase 0.1%
formula 9: glucose alone
formula 10: glucose+0.1% glucose oxidase
formula 11: glucose+ZnO 1%
formula 12: glucose+0.1% glucose oxidase+ZnO 1%

The microbial strains used for the tests are the following bacteria:
P. aeruginosa
S. aureus

The tests were conducted on microplates to evaluate the MIC (Minimum Inhibitory Concentration).

a. Bacterial Proliferation Tests

The bacterial proliferation in the presence of the products to be tested is determined using a 96 well microplate method. Each well is inoculated with 50 μL of bacterial suspension to be tested in a Muller-Hinton (MH) broth+150 μL of diluted test product solution. The bacterial concentration in the well is set at 10⁶ CFU/mL.

The positive control (50 μL of bacterial suspension+150 μL of MH medium) corresponds to 100% bacterial proliferation. The negative control (200 μL of culture medium) corresponds to 0% bacterial proliferation.

Each specimen is tested in triplicate. The optical densities (OD) are read a first time at time 0 (T0) at 450 nm. The plates are then incubated for 24 h at 37° C. with agitation. At the end of incubation, the OD is measured again at 450 nm (T24). The proliferation percentage is calculated as follows:

Proliferation=(DO_{T24}−DO_{T0})/(DO_{T24Control+}−DO_{T0Control+})

The results obtained are shown in tables 1 and 2 below:

TABLE 1
Influence of active ingredients used and formulas developed from those
active ingredients on the proliferation of P aeruginosa. The formulas
are tested on microplates at concentrations of 1.5%, 3%, 6% and 9% (v/v).
The proliferation percentages are calculated from the positive control
corresponding to the untreated medium
(culture medium alone = 100% proliferation)

| Tested formulas | 1.5% | 3% | 6% | 9% |
|---|---|---|---|---|
| Formula 1 | 92% | 98% | 48% | 6% |
| Formula 2 | 76% | 86% | 40% | −7% |
| Formula 3 | 119% | 125% | 99% | 55% |
| Formula 4 | 107% | 107% | 39% | −4% |
| Formula 5 (invention) | 72% | 30% | −4% | −11% |
| Formula 6 (invention) | 64% | 44% | −31% | −30% |
| Formula 7 (invention) | 86% | 41% | −14% | −15% |
| Formula 8 | 96% | 96% | 91% | 86% |
| Formula 9 | 100% | 206% | 206% | 113% |
| Formula 10 | 96% | 98% | 89% | 79% |
| Formula 11 | 156% | 86% | 60% | 41% |
| Formula 12 (invention) | 94% | 63% | 31% | 0% |

One can see that formulas 5, 6, 7 and 12 cause a sharp decrease in the proliferation of P. aeruginosa. This decrease is much greater for all of the formulas containing at least one carbohydrate, GOD and ZnO compared to the formulas containing at least one carbohydrate and GOD (formulas 2 and 10) or at least one carbohydrate and ZnO (formulas 4 and 11). For formula 12, the bacterial proliferations decrease by 79% and 41%, respectively, at the concentration of 9% v/v to reach the MIC. The MIC goes from 9% v/v to 6% v/v for formulas where the carbohydrate source is honey.

TABLE 2
Influence of active ingredients used and formulas developed from those
active ingredients on the proliferation of S. aureus. The formulas are
tested on microplates at concentrations of 1.5%, 3%, 6% and 9% (v/v).
The proliferation percentages are calculated from the positive control
corresponding to the untreated medium
(culture medium alone = 100% proliferation)

| Tested formulas | 1.5% | 3% | 6% | 9% |
|---|---|---|---|---|
| Formula 1 | 96% | 49% | −4% | −29% |
| Formula 2 | 75% | 37% | 7% | −34% |
| Formula 3 | 109% | 117% | 105% | 75% |
| Formula 4 | 90% | 17% | −13% | −44% |
| Formula 5 (invention) | −2% | 4% | 3% | −30% |
| Formula 6 (invention) | 4% | −17% | −10% | −58% |
| Formula 7 (invention) | 7% | −4% | −37% | −57% |
| Formula 8 | 81% | 72% | 54% | 46% |
| Formula 9 | 96% | 86% | 83% | 72% |
| Formula 10 | 92% | 95% | 72% | 65% |
| Formula 11 | 78% | 25% | 8% | −8% |
| Formula 12 (invention) | 52% | 2% | −23% | −53% |

One can see that formulas 5, 6, 7 and 12 cause a sharp decrease in the proliferation of S. aureus. This decrease is much greater for all of the formulas containing at least one carbohydrate, GOD and ZnO compared to the formulas containing at least one carbohydrate and GOD (formulas 2 and 10) or at least one carbohydrate and ZnO (formulas 4 and 11). For formula 12, the bacterial proliferations decrease by 95% and 25%, respectively, at the concentration of 3% v/v to reach the MIC. The MIC goes from 9% v/v to 6% v/v for formulas where the carbohydrate source is honey.

b. Anti-Biofilm Activity

The anti-biofilm activity in the presence of the products to be tested is determined using a 96 well microplate method. Each well is inoculated with 50 μL of bacterial suspension to be tested in a tripticase soy broth+1% glucose (TS+1%Glu)+150 μL of diluted test product solution. The bacterial concentration in the well is set at 10⁶ CFU/mL. The positive control (50 μL of bacterial suspension+150 μL of TS+1%Glu medium) corresponds to 100% bacterial proliferation. The negative control (200 μL of culture medium) corresponds to 0% bacterial proliferation. Each specimen is tested in triplicate. The plates are next incubated for 24 h at 37° C. At the end of incubation, the supernatant is eliminated, the microplate is washed 3 times with 200 μL/well of distilled water. The plate is then incubated in a drying apparatus at 60° C. for 30 minutes. A 0.5% crystal violet (CV) solution is added and the plate is incubated for 20 minutes at ambient temperature. At the end of incubation, the CV solution is removed and the microplate is washed 3 times with 200 μL/well of distilled water. The plate is then dried overnight at 60° C., and lastly 100 μL/well of a 33% acetic acid solution is added into the wells. The plate is then photographed. The wells in which the biofilm has formed are violet (+), while the wells in which no biofilm has formed are clear (−).

The results obtained are shown in tables 3 and 4 below:

TABLE 3
Influence of active ingredients used and formulas developed from
those active ingredients on the proliferation of P aeruginosa.
The formulas are tested on microplates at concentrations of
1.5%, 3%, 6% and 9% (v/v). The biofilms are revealed using a
0.5% crystal violet solution. The positive control corresponds to
the culture medium + P. aeruginosa control; the negative
control corresponds to the culture medium without bacteria

| Tested formulas | 1.5% | 3% | 6% | 9% |
|---|---|---|---|---|
| Formula 1 | + | + | + | − |
| Formula 2 | + | + | + | − |
| Formula 3 | + | + | + | + |
| Formula 4 | + | + | + | − |
| Formula 5 (invention) | + | + | − | − |
| Formula 6 (invention) | + | + | − | − |
| Formula 7 (invention) | + | + | − | − |

One can see a decrease in biofilm activity at 6% v/v with the {honey+glucose oxidase+ZnO} combination on P. aeruginosa.

TABLE 4
Influence of active ingredients used and formulas developed from
those active ingredients on the proliferation of S. aureus. The
formulas are tested on microplates at concentrations of 1.5%, 3%,
6% and 9% (v/v). The biofilms are revealed using a 0.5% crystal
violet solution. The positive control corresponds to the culture
medium + S. aureus control; the negative control
corresponds to the culture medium without bacteria

| Tested formulas | 1.5% | 3% | 6% | 9% |
|---|---|---|---|---|
| Formula 1 | + | + | − | − |
| Formula 2 | + | + | − | − |
| Formula 3 | + | + | + | + |
| Formula 4 | + | + | − | − |
| Formula 5 (invention) | − | − | − | − |
| Formula 6 (invention) | − | − | − | − |
| Formula 7 (invention) | − | − | − | − |

One can see a decrease in anti-biofilm activity at 1.5% v/v with the {honey+glucose oxidase+ZnO} combination on S. aureus.

Demonstration of the Synergistic Effect

Principle of the Berenbaum Test

The Berenbaum test makes it possible to determine the additive, synergistic or antagonistic effects between two products placed in mixtures in contact with cells (Berenbaum, 1977). This test was developed in order to determine the impact of several mixed compounds using experimental data inserted into a mathematical formula. It is applicable to many areas of biology. Here, this test is applied to {honey+GOD} and {ZnO} from the bacterial proliferation results obtained on the S aureus and P aeruginosa strains.

The proliferation results are analyzed based on a search for equivalent activity for each compound. For example, the activity sought is a proliferation of 50%. The equivalent active dose necessary to obtain 50% proliferation (in the case of our example) is designated by A_{eq} for {honey+GOD}. B_{eq} designates the equivalent dose necessary to obtain the same effect (50% proliferation) with {ZnO}.

Secondly, the mixture of the two compounds makes it possible to determine the doses of each product to be introduced together to obtain the same effect (50% proliferation) as with A_{eq} or B_{eq}. The necessary dose of {honey+GOD} is then designated as A, and that of {ZnO} as dose B.

Subsequently, it is possible to introduce this data into the following equation:

$$Indice de Berenbaum = \frac{A}{{A}_{éq}} + \frac{B}{{B}_{éq}}$$

If the result of this equation is equal to 1, the effect of the 2 compounds will be additive.
If the result is less than 1, synergy exists.
However, if the result is greater than 1, the compounds are designated antagonistic.

Results of the Berenbaum Test

TABLE 5
Necessary active doses to obtain bacterial proliferation of 50%.

| FORMULAS | S aureus | P aeruginosa |
|---|---|---|
| Formula 2 | A_{eq} = 6.10⁻³% | A_{eq} = 3.10⁻³% |
| Formula 3 | B_{eq} = 9.10⁻²% | B_{eq} = 9.10⁻²% |
| Formula 5 (invention) | A = 3.10⁻³% | A = 1.5, 10⁻³% |
| | B = 3.10⁻²% | B = 1.5, 10^{-2\}% |
| Berenbaum index | 0.8 | 0.7 |

The Berenbaum index is less than 1 for the 2 bacterial strains. The formula according to the invention therefore shows a synergistic action of the active compounds.

2. Comparison of the Antibacterial Efficacy with Honey having a Peroxide Activity

Tests have been performed with honey exhibiting an equivalent peroxide activity (peroxide activity of 0.025 μg/g of honey per hour) to which zinc oxide, with or without glucose oxidase has been added.

The operating protocolis the same as point 1 a).

The results obtained on the proliferation of Pseudomonas aeruginosa in the presence of different compositions of honey diluted to a concentration of 6%, are presented in table 5a below:

TABLE 5a
importance of the presence of GOD
to obtain an effect on bacterial proliferation

| GOD | ZnO | % bacterial proliferation |
|---|---|---|
| 0 U/g | 0% | 126% |
| 0 U/g | 0.5% | 85% |
| 0 U/g | 1% | 58% |
| 0 U/g | 2% | 62% |
| 0.5 U/g | 1% | 0% |
| 5 U/g | 1% | 0% |
| 0.5 U/g | 2% | 0% |
| 5 U/g | 2% | 0% |

It is found that the sole peroxide activity of honey is insufficient to obtain the antibacterial activity and that the presence of glucose oxidase is necessary to obtain a synergistic effect.

3. Cytotoxicity Tests

In order to evaluate the cytotoxicity of the formulas, a cell count test in the presence of honey and a composition according to the invention (formula 6) was done on fibroblasts.

Protocol

The products are diluted to obtain a concentration of 5.12% v/v of the specimen.

The operating mode is as follows:

The primary cultures of PAR 08052 human fibroblasts are next seeded in 48 well microplates at a rate of 10,000 cells per well with a whole culture medium volume of 500 μL. The cells will next adhere to the bottom of the wells for 24 hours. The medium will next be replaced by the product to be tested, diluted in the culture medium without serum. The product is tested at a rate of 500 μL per well and incubated for 48 hours at 37° C.+5% CO2. The cells are next taken off with trypsin, then counted on Malassez slides with an exclusion dye (Trypan blue). The fibroblast proliferation percentage is determined relative to a control not treated with the composition.

Results

The results are shown in table 6 below.

TABLE 6
Cell count on fibroblasts with honey-based formulas. The control medium
corresponds to the culture medium, which alone was used to dilute the
specimens (dilution in % v/v). The cells were treated for 48 h.

| | Control | | |
|---|---|---|---|
| Specimens | medium | Honey | Formula 6 |
| Fibroblast proliferation | 100% +/− 21% | 100% +/− 22% | 88% +/− 22% |
| percentage at 1.28% | | | |
| (%) | | | |

The composition according to the invention (formula 5) does not have a significant cytotoxicity.

4. In Vivo Efficacy

The in vivo efficacy of the composition according to the invention was evaluated in a panel of patients treated in a care structure (retirement home, hospitals).

Formula 5 {Honey+GOD 0.1%+ZnO 1%+Excipients} was tested in 6 patients with wounds in the cleaning phase, fibrous (n=6) and/or necrosed (n=4), infected (n=1) or not (n=5).

The results of the study are shown in table 7 below:

TABLE 7

| | | | | Efficacy on | | Necrosis | | | | Comparative |
|---|---|---|---|---|---|---|---|---|---|---|
| | | | | colonization | | efficacy | | Inflammation | | assessment |
| | | | | Not | | Not | | efficacy | | with honey |
| | | | | effective/ | | effective/ | | Not effective/ | | alone |
| | | | Initial | Mildly | | Mildly | | Mildly | | Not at all |
| | | | bacterial | effective/ | | effective/ | | effective/ | | effective/ |
| | | Fibrin | colonization | Effective/ | | Effective/ | | Effective/ | | Less |
| | | efficacy | Colonization/ | Highly | | Highly | | Highly | Improved | effective/ |
| | | Poor/Fair/ | Infection/ | effective/ | | effective/ | Inflam- | effective/ | healing | Equivalent/ |
| Type of | Fibrin | Good/Very | lack of | Not | Necrosis | Not | mation | Not | speed | More |
| wound | Yes/No | good | contamination | applicable | Yes/No | applicable | Yes/No | applicable | Yes/No | effective |
| Mixed | Yes | Very good | Lack of | Not | No | | No | Not | Yes | More |
| ulcer | | | contamination | applicable | | | | applicable | | effective |
| Heel | Yes | Good | Lack of | Not | Yes | Effective | No | Not | Yes | More |
| eschar | | | contamination | applicable | | | | applicable | | effective |
| Sacral | Yes | Good | Lack of | Not | Yes | Highly | No | Not | Yes | More |
| eschar | | | contamination | applicable | | effective | | applicable | | effective |
| Eschar | Yes | Fair | Infection | Effective | Yes | Effective | No | Effective | Yes | More |
| | | | | | | | | | | effective |
| Eschar | Yes | Good | Lack of | Not | Yes | Effective | Yes | Effective | Yes | More |
| ulcer | | | contamination | applicable | | | | | | effective |
| ulcer, | Yes | Very good | Lack of | Effective | No | | Yes | Effective | Yes | More |
| eschars | | | contamination | | | | | | | effective |

One can see:
83% efficacy on fibrin
Efficacy on infected wound
100% efficacy on necrosis
100% efficacy on inflammation
100% efficacy on improving healing speed
100% of the tested panel found that the tested formula is more effective for cleaning than honey alone.

1. A composition comprising a mixture made up of at least one carbohydrate, glucose oxidase and zinc oxide, the glucose oxidase being present in an amount of at least 0.025 mU per gram of composition.

2. The composition according to claim 1, wherein the mixture made up of at least one carbohydrate, glucose oxidase and zinc oxide contains between 1 and 99 wt % of carbohydrate relative to the total weight of the composition.

3. The composition according to claim 1, wherein the mixture made up of at least one carbohydrate, glucose oxidase and zinc oxide contains between 0.5 mU and 50 U of glucose oxidase per gram of composition.

4. The composition according to claim 1, wherein the mixture made up of at least one carbohydrate, glucose oxidase and zinc oxide contains between 0.1 and 40 wt % of zinc oxide relative to the total weight of the composition.

5. The composition according to claim 1, wherein the at least one carbohydrate is glucose.

6. The composition according to claim 1, wherein the mixture is made up of honey, glucose oxidase and zinc oxide, said honey comprising said at least one carbohydrate.

7. The composition according to claim 6, wherein the honey is natural honey.

8. The composition according to claim 6, wherein the honey is selected from the group consisting of thyme honey, honeydew honey, buckwheat honey, manuka honey, and mixtures thereof.

9. The composition according to claim 6, wherein the honey is artificial honey or a mixture of natural honey and artificial honey.

10. The composition according to claim 9, wherein the artificial honey is primarily made up of a carbohydrate selected from the group consisting of glucose, fructose, sucrose and combinations thereof.

11. The composition according to claim 1, further comprising at least one component selected from the group consisting of vanillin, caprylylglycol, pentylene glycol, 1,2-hexanediol, a propolis extract, petroleum jelly, paraffins, lanolin, pectins, starch or derivatives of starch, alginate or derivatives of alginates, chitosan or derivatives of chitosan, cellulose or derivatives of cellulose, gums, carrageenans, xanthanes, beta-glucans, methylglyoxal, synthesis polymers, waxes, natural or artificial oils, butters, mineral products, glycerides and other fatty esters, surface active agents, water, ethanol, propylene glycol, polyethylene glycols, butylene glycol, glycerol, sorbitol, extracellular matrix compounds, hydrocarbons and silicones, proteins and peptides.

12. The composition according to claim 1, wherein the imposition is in a form selected from the group consisting of a powder, a liquid and a semi-liquid.

13. The composition according to claim 1, wherein the composition is in a form selected from the group consisting of a powder for cutaneous use, a spray, gel, pomade, cream, emulsion, suppository and vaginal suppository.

14. The composition according to claim 1, wherein the composition has been treated by gamma radiation.

15. A topical antimicrobial product comprising the composition of claim 1.

16. A topical healthcare product comprising the composition of claim 1.

17. A topical anti-inflammatory healthcare product comprising the composition of claim 1.

18. A health product to treat cutaneous irritations or lesions, comprising the composition of claim 1.

19. A health product to treat burns, post-operative scar disunions, residual cavities of the pilodinal cavities, surgical scars infected after flattening, ulcers and eschars, dermabrasions, traumatic and/or surgical wounds, chronic wounds, acute wounds, cancerous wounds, ostomy locations or superficial wounds, comprising the composition according to claim 1.

20. A health product to treat scratch dermatitis, diaper rash, acne, dermatitis, boils, folliculitis, whitlow, mycosis or impetigo, comprising the composition according to claim 1.

21. The composition according to claim 1, wherein the composition is in a form selected from the group consisting of a vaginal gel to treat cutaneous lesions and microbial infections, a rectal cream or suppository to treat rectal lesions and irritations, a cream for the hands and feet for cracking and chilblains, a balm to prevent and/or treat cracked nipples, an oral gel to treat canker sores and mucositis, and a lip stick or balm to treat cracked skin.
